(19) United States
(12) Patent Application Publication
(10) Pub. No.: US 2007/0249531 A1
(43) Pub. Date: Oct. 25, 2007

(54) Method for the Detection of Autoantibodies Against Specific Peptides and Its Use in Diagnosis and Treatment of Pregnancy-Loss or Infertility

(75) Inventors: Hans-Georg Frank, El Kerkrade (NL); Andreas Rybka, Herten (DE); Andrea Borges, Maplewood, NJ (US); Alexandra Greindl, Langerwehe-Merode (DE)

Correspondence Address: VENABLE LLP, P.O. BOX 34385, WASHINGTON, DC, 20043-9998, US

(73) Assignee: AplaGen GmbH, Bacsweiler (DE)

(21) Appl. No.: 10/598,153

(22) PCT Filed: Feb. 21, 2005
(86) PCT No.: PCT/EP05/50742

§ 371 Date: Feb. 28, 2007

Related U.S. Application Data
(60) Provisional application No. 60/545,906, filed on Feb. 20, 2004.

(51) Int. Cl. A61K 38/00; C07K 14/00; C12N 5/06; G01N 33/53

(52) U.S. Cl. 514/12; 435/7.8; 436/547; 530/300; 530/324

(57) ABSTRACT

A method of assessing the titre of fertility-relevant autoantibodies of a mammalian female, wherein said method comprises: the presentation of a defined single molecule capable of binding to the idiotype of the fertility-relevant autoantibody, and determining the amount of autoantibody being bound by said molecule from a sample of a body fluid of said mammalian female.

### TECHNICAL FIELD AND BACKGROUND OF THE INVENTION

The present invention relates to synthetic peptides as well as to proteins related to these peptides and to pharmaceutical compositions comprising the peptides/proteins for the diagnosis and treatment of fertility-relevant and/or pregnancy-relevant autoantibodies.

Autoimmune diseases are disorders in which the immune system erroneously produces autoantibodies characterized by their binding to an endogenous antigen, with subsequent partial or complete loss of function of the endogenous antigen. Loss of function and other pathogenetic events are due to a number of effects, which can be associated with the binding of an antibody to an endogenous antigen:

Functionally relevant parts of the self-antigen (e.g. active enzymatic sites, binding areas, etc.) can be blocked by the antibody.

The turnover of the protein can be increased and the active concentration be decreased

Antigen-Antibody complexes can be deposited and trigger pathogenetic pathways.

Compartmentalisation of the antigen can be influenced.

Bound antibody can trigger a number of responses in the immune system, which secondarily influence the antigen itself or other parts of the body. Other autoantibody-mediated mechanisms might be possible, too. In addition, cell-mediated immune responses can contribute to the overall pattern of symptoms observed in a given autoimmune disease.

Several connective tissue disorders including vascular diseases such as vasculitis, systemic lupus erythematosus (SLE) and polymyositis, neurologic diseases such as multiple sclerosis and myasthenia gravis, and hematologic diseases such as idiopathic thrombocytopenic purpura (ITP) and anti-phospholipid syndrome (APS) seem to be caused by an autoimmune reaction. For some of these disorders, possible self antigens have been identified and/or pathogenic autoantibodies have been identified and isolated.

However, no specific drugs exist nowadays for the treatment of autoimmune diseases and patients are treated with general anti-inflammatory drugs such as corticosteroids and/or symptom-specific drugs like anticoagulants (McIntyre et al. 2003) in the case of coagulation disorders due to autoantibodies.

Moreover, for many of the fertility-relevant autoantibody syndromes, such as SLE and APS, the antigens/antibodies causing the reproductive problems are not identified and the correlation between the general diagnostic detection of e.g. APS-autoantibodies and severity of reproductive symptoms is poor (Beer et al. 1998; Bermas et al. 1996a; Bermas et al. 1996b, 1996c; Bick and Baker 1999; Birdsall et al. 1996; Check 1998; Chilcott et al. 2000; Colaco and Male 1985). This is partly due to the fact, that most approaches to fertility-relevant autoantibodies did start with one of these broad polyclonal antibody syndromes like APS and identified prominent autoantibodies, which did not necessarily cause the reproductive problem, but were either representative for the whole syndrome or associated with another part of the syndrome.

It is an object of the invention to provide means for diagnosis of autoantibodies and specific treatment of autoantibody-related symptoms, which are associated with and/or cause reproductive problems.

The present invention relates to novel peptides and proteins related to these peptides, which are anti-idiotypic to fertility-relevant autoantibodies. Thereby, the invention overcomes the lack of fertility-specific diagnostic and/or therapeutic means in the field of reproductive autoimmunity.

### DESCRIPTION OF THE INVENTION

General Description

Surprisingly, the problem underlying the invention is solved by methods, peptides, diagnostics, medicaments and kits of claims 1 to 19.

Peptides, which are able to bind autoantibodies being present in the blood of patients with reproductive problems, are obtainable by the following method:

A large number of biotinylated combinatorial peptides was synthesized by solid phase synthesis.

These peptides were tested against plasma probes of patients with well-defined clinical symptomotology as well as against control samples.

Peptides with specific recognition of patient samples (recognition defined by increased binding of human antibodies to the peptide as compared to the control samples) were tested in a larger population of samples.

Sequence variations of each positive peptide were tested to optimise a positive sequence.

Peptide sequences were used to check databases for sequence-related proteins harbouring the autoantibody target.

Surprisingly, so far unknown peptides could be identified, which matched on fertility-relevant proteins.

The medicament and the diagnostic of the invention are useful for the treatment and diagnosis of fertility disorders or pregnancy complications.

Subject of the invention are also pharmaceutical compositions comprising the peptides of the invention, including variants such as derivatives or multimers, preferably in a suitable formulation for contraception or the induction of sterility in a patient, caused by the generation of an antibody response to the respective peptide.

### DETAILED DESCRIPTION

More than thousand random 12-mer peptide sequences were synthesized on an automated synthesis roboter and purified on an automated LC/MS-System, which enables the purification and analysis in a one-step approach. All sequences were foreseen with an additional terminal Glycine residue coupled to a biotin molecule. Per peptide, an amount of 5-10 mg—the final yield per specific peptide depending on the losses due to purification and problematic synthesis—were synthesized. A typical synthesis protocol is given as an example of the invention.

Plasma samples were obtained from patients with clinically well-documented reproductive problems. Samples from healthy female blood donors were used as reference.

Non-immunological causes (anatomical variations, endocrine problems, fertility problem of the male, etc.) of the respective reproductive disorders present in a given patient, were excluded. Moreover, all plasma samples were checked for the presence of anti-phospholipid antibodies. APS is an autoimmune syndrome, which is frequently associated with coagulation disorders, but also with fertility problems. Samples with anti-phospholipid antibody titres above the clinically relevant threshold were excluded from the first step of the peptide screen. Plasma samples were grouped according to the most common reproductive disorders:

Unexplained infertility in patients with repetitive (at least 2 times) failure in conventional in vitro-fertilization procedures.

Habitual abortion, characterized by at least 3 consecutive abortions during the 11 trimester of pregnancy.

Late pregnancy problems, this group being heterogenous and composed of plasma samples from patients with preeclampsia and/or severe intrauterine growth restriction of the fetus without maternal preeclamptic complications.

In an ELISA-protocol, the serum and plasma samples were tested against peptides from the available peptide pool. Basically, the ELISA was designed to detect antibodies being present in the patients blood, which bind to the peptides presented to the sample. Technically, the ELISA followed the protocol given in Example 2 and comprised the following basic steps:

Binding of biotinylated peptides on streptavidin-coated Microtitre (96-well) plates.

Washing and blocking of the wells.

Incubation of the wells and appropriate control wells (without peptide) with the serum samples.

Washing.

Incubation with a suitable horseradish-peroxidase-labelled secondary antibody

Chromogenic detection and photometric read-out of the results.

In a first step, all peptides were tested against a reduced set of samples including pooled sample preparations in order to identify promising candidate peptides for large scale analysis. In a second step, promising candidate peptides were tested against all samples to confirm the results obtained in the 1^{st} screen. Using such ELISA-based protocols, it is easily possible to examine large numbers of blood samples and to test peptides against such samples. We could identify the following peptides, which showed clearly increased binding of patient's antibodies as compared to control antibodies:

The Peptide 1 VYKSPNAYTLFS

The Peptide 2 RPEPQGAYLEQG

The Peptide 3 NSSYSPSLLESG

The Peptide 4 DQYIQQAHRSHI

The Peptide 5 QGLPAPQSYSRI

The Peptide 6 KQASNLTDMHYP

The Peptide 7 AQPNWTSLRSLP

The Peptide 8 HVNPHLHVHAWD

Based on these peptides, it is easily possible to deduce sequence variants, which also have the ability to bind antibodies occurring in the tested samples. For the skilled person in the field it is evident that conservative exchange of amino acids in the above mentioned peptide sequences will also lead to binding peptides, if the exchange does not affect the physicochemical or structural properties of the peptide sequence to a large extent.

In further embodiments of the invention oligopeptides are used which are derivatives, fragments and/or homologues of the oligopeptides of Seq. ID No. 1 to 8.

They differ from the oligopeptides such that
the oligopeptide amino acid sequence is extended at the amino-terminus and/or the carboxy-terminus by either up to 5 amino acids per terminus, preferably by up to 3 or 2 amino acids and/or
in the homologous oligopeptides up to 3, preferably 2 or 1 amino acids are substituted by other amino acids, and/or
the fragments lack 1 or 2 amino acids at the N- and/or C-terminus, and/or
the oligopeptides are derivatized for detection by modifications including biotinylation, labelling by fluorochromes or radiolabelling.

The oligopeptides of the invention comprise Retro-Inverso derivatives of these peptides, derivatives, fragments or homologues.

In a further embodiment of the invention, the oligopeptides are fragments of the sequences of pregnancy-associated plasma protein A (PAPP-A; gi:38045915) or from ADAM-TS 13 (gi:21265049) comprising 8 to 50, preferably 10 to 40 or 10 to 20 amino acids.

An especially preferred way of intensifying the reactivity of the above mentioned peptides with autoantibodies is dimerisation or even multimerization. Surprisingly, we could demonstrate that biotinylated homodimers of these peptides, connected by a disulfide bridge after elongation with a cysteine residue, strongly increase the signals which can be detected. Increased reactivity of dimers as compared with the monomeric peptides is also shown in FIG. 2. Dimerisation is thus proven to effectively increase the signal intensity, and it is obvious that other technically suitable ways of generating di- or multimeric peptides based on said peptides as reactive moieties also lead to increased signal intensity. Thus, dimers, trimers or multimers of the above mentioned peptides are subject of the invention.

Using the peptide sequences as search sequences in B LAST-algorithms as they are offered e.g. by the NCBI or EMBL Web Portals ended up with proteins, which harbour sequences similar or identical to the peptides 1-3.

Suprisingly good results were obtained with sequences 1 and 2 with Pregnancy-associated plasma protein A (Papp-A). Both peptides show varying degrees of homology with this protein sequence.

Peptide Sequence 3 shows homology with the Protein ADAM-TS 13 (a disintegrin and a metalloproteinase and thrombopondin-13).

Peptide Sequence 1 shows a very good correlation with a sequence occurring in Pregnancy-associated Plasma Protein A (PAPP-A). Peptide Sequence 2 shows homologies with another region in PAPP-A.

Amino acids described in this invention can be of the naturally occurring L stereoisomer form as well as the enantiomeric D form. The one-letter code refers to the accepted standard polypeptide nomenclature, but can mean alternatively a D- or L-amino acid:

Code amino acids

A L-Alanine or D-Alanine

V L-Valine or D-Valine

L L-Leucine or D-Leucine

I L-Isoleucine or D-Isoleucine

M L-Methionine or D-Methionine

F L-Phenylalanine or D-Phenylalanine

Y L-Tyrosine or D-Tyrosine

W L-Tryptophan or D-Tryptophan

H L-Histidine or D-Histidine

S L-Serine or D-Serine

T L-Threonine or D-Threonine

C L-Cysteine or D-Cysteine

N L-Asparagine or D-Asparagine

Q L-Glutamine or D-Glutamine

D L-Aspartic acid or D-Aspartic acid

E L-Glutamic acid or D-Glutamic acid

K L-Lysine or D-Lysine

R L-Arginine or D-Arginine

P L-Proline or D-Proline

G Glycine

### EXAMPLES OF THE INVENTION

Synthesis of Peptides, Example

General Comments:

The Protocol below describes the general setup for synthesis of the specific sequences 1-8 described above. For proper usage in the ELISA-protocol on Streptavidin-coated microtitre plates, these sequences are all extended N-terminally by one glycine moiety. The N-term of this glycine moiety is coupled to biotin in the last step of synthesis.

In order to prepare dimeric peptides, the sequences are N-terminally extended by a, preferably the sequence GCG, with the last glycine moiety either being biotinylated N-terminally or not. Equimolar amounts of biotinylated and not-biotinylated compounds are then mixed together and oxidized in order to achieve dimerisation by closure of disulfide bridges. Purification by HPLC-MS then is used to isolate those dimers, which harbour one biotinylated and one not-biotinylated peptide moiety. Such monovalently biotinylated dimers are then used for the assay as their mono-valently biotinylated monomeric analogs.

Unless stated otherwise a washing step is conducted by adding the solvent to the resin, shaking the mixture, and removing the solvent by vacuum filtration. At all steps it must be ensured that each resin bead is immersed in the reaction solution.

Step 1: Loading of the Resin with the First Amino Acid

1 g 2-chlorotrityl resin (1.0-2.0 mmol/g capacity) is suspended in 8 ml dichloromethane (DCM), shaken for 5 minutes at room temperature, and the solvent is removed by vacuum filtration. A solution of 2 mmol of the FMoc protected amino acid and 5 mmol (0.850 ml) diisopropylethylamine (DIPEA) in 8 ml DCM is added and the reaction is shaken for 1 hour at room temperature. After removing the reaction solution the resin is washed three times with 20 ml dimethylformamide (DMF) each. 20 ml of a mixture of DCM/Methanol/DIPEA 80:15:5 (v/v/v) is added, shaken for 15 to 30 minutes, the solution removed, and this step is repeated once. The resin is washed four times with 20 ml DMF each. The FMoc group is removed by adding 20 ml of 25 vol-% piperidine in DMF, shaking for 3 minutes, removing the solvent, adding another 20 ml of 25 vol-% piperidine in DMF, shaking for 30 minutes, and removing the solution by vacuum filtration. The resin is washed six times with 20 ml DMF each. In this state the resin can be stored overnight. For this purpose it has to be washed two times with 20 ml DCM each, and dried in vacuo. Should a second amino acid be coupled the procedure can be directly continued at step two instead of washing with DCM.

Step 2: Coupling of the 2nd Amino Acid

In the case that the resin has been stored overnight it has to be swollen by filling the reaction vessel completely with DMF. After 20 minutes the DMF is removed.

A solution of 5 mmol of the FMoc protected amino acid, that will be introduced, 7.5 mmol 1-hydroxybenzotriazole (HOBt), and 1 ml of DIPEA in 20 ml DMF (eventually up to 30 ml in the case that the amino acid derivative is not dissolved completely) is added to the resin. The suspension is vortexed for 5 minutes and 5 mmol of benzotriazole-1-yl-oxy-trispyrrolidinophosphonium hexafluorophosphate (PyBOP)-is-added as a solid as well as another ml of DIPEA. After vortexing for 60 to 90 minutes the reaction solution is filtered off, and the resin is washed 6 times with 30 ml DMF each. The resin can be stored in this state (after washing twice with DCM and drying in vacuo).

Step 3: Coupling of Further Amino Acids or Biotin

Further amino acids are coupled by removing the FMoc group with 25% piperidine in DMF (as described above) and repeating Step 2. Biotin is introduced by repeating step 2 using biotin instead of an amino acid derivative. Due to the poor solubility of biotin the coupling time was four times as long as for a normal amino acid.

Step 4: Cleaving the Peptide Off the Polymer

The resin which is loaded with the FMoc deprotected peptide is washed 6 times with 20 ml DMF and twice with 20 ml DCM each. 40 ml 2,2,2-trifluoroethanol/DCM 2:8 (v:v) is added. The reaction mixture is shaken from time to time and otherwise left standing for 60 minutes. The resin is filtered off and the filtrate co-evaporated several times with DCM.

Step 5: Deprotecting the Peptide

40 ml of a mixture of trifluoroacetic acid/water/triisopropylsilane (TIS) 95:5:5 (v/v/v) is added. In the case that the solution is still coloured yellow after about 1 minute several drops of TIS are added. The mixture is left standing for about 60 minutes. Afterwards the cleaving mixture is removed by coevapourating several times with DCM. The product is dissolved in water (eventually adding a minimal amount of methanol) and lyophilisated. The crude peptide is purified by preparative HPLC.

Step 6: Synthesis of a Heterodimer

Two peptides with the same amino acid sequence but different derivatisation of the N-termini are oxidized to form a heterodimer. For that purpose 10 mg each of the biotinylated and the non-biotinylated peptide (HPLC purified) are dissolved in 200 ml acetonitrile/water (1:1). 10 ml dimethylsulfoxide are added and the reaction solution is vortexed for 10 hours at room temperature. The solvent is removed as far as possible from the reaction mixture by vacuum distillation, the remaining solution is lyophilisated and the residue is purified by preparative HPLC to isolate the heterodimer.

Example of an ELISA-Protocol

Materials Used in the Protocol

96well streptavidin plates from Steffens (Steffens GmbH, Heidelberg, Germany)

Biotinylated IgG from Reactolab SA (5 mg/ml)

Anti-human IgA, IgG, IgM from Sigma A8794 (22 mg/ml)

Shaking at all incubation and washing steps was done on a laboratory shaking platform at 150 rpm

Dilute APL peptides from (dimethylsulfoxide) DMSO stock (10 mg/ml) into washing buffer to 1 μg/ml (for this dilution, pipette: 1 μl in 10 ml)

Dilute stock of Reactolab solution 1:1000 in washing buffer +2% Milk powder +1% FCS, then:

For wells A 1,2: pipette 100 μl washing buffer +2% Milk powder +1% FCS into each well.

For wells A 3,4: pipette 4 μl of this solution into 2 ml washing buffer +20% Milk powder +1% FCS, then pipette 100 μl of this solution into each well.

For wells A 5,6: pipette 1011 of this solution into 2 ml washing buffer +2% Milk powder +1% FCS, then pipette 100 μl of this solution into each well.

For wells A 7,8: pipette 20 μl of this solution into 2 ml washing buffer +2% Milk powder +1% FCS, then pipette 100 μl of this solution into each well.

For wells A 9,10: pipette 30 μl of this solution into 2 ml washing buffer +2% Milk powder +1% FCS, then pipette 100 μl of this solution into each well.

For wells A 11,12: pipette 40 μl of this solution into 2 ml washing buffer +2% Milk powder +1% FCS, then pipette 100 μl of this solution into each well.

| Step | Standard (Row A) | Peptide and LW (B-H) |
|---|---|---|
| 1 | Equilibrate all wells in A | Equilibrate all wells (B-H) with 100 μl |
| | (calibration curve*) with 100 μl | washing buffer (10 min., Room |
| | washing buffer + 2% milk | Temperature) |
| | powder + 1% FCS (10 min., | |
| | Room Temperature) | |
| 2 | Pipette 100 μl of the | Pipette 100 μl of the diluted APL peptides |
| | appropriately diluted standard | (100 ng total amount) in the wells, last |
| | solution into the respective wells | row (H, containing Blank Values) without |
| | and incubate 1 hour at RT. | peptide (only washing buffer), incubate 1 |
| | | hour at RT |
| 3 | Wash 3x 5 min. with washing buffer | |
| 4 | Add 100 μl washing buffer per | Add 1% human serum or plasma diluted |
| | well, incubate 1h RT | in blocking buffer, 100 μl per well, |
| | | incubate 1h RT |

| | |
|---|---|
| 5 | Wash 3x 10 min. with washing buffer |
| 6 | Add 100 μl anti-hu IgA,G,M-HRP1:10000 |
| | (Stock: 22 mg/ml) diluted in blocking buffer |
| | to all wells, incubate 1 hour at RT |
| 7 | Wash 3x 5 min. with washing buffer |
| 8 | Add 100 μl OPD substrate to each well: 1 tablet |
| | OPD (20 mg) in 33 ml Citrate-phosphate buffer + |
| | 17 μl 30% H2O2 (suffices |
| | for 3 plates) |
| 9 | Incubate OPD for 10 min and stop reaction with 100 μl |
| | 1N HCL to each well |
| 10 | Measure at measuring WL: 492 with reference WL: 620 in |
| | ELISA reader |

Washing Buffer:

10 mM Tris, pH7.5 (10 ml IM Tris, pH7.5)

2,1% NaCl NaCl (21 g)

2 mM EDTA (4 ml 0.5M EDTA)

1 ml TritonX-100

ad aq. dest. to 1|

Citrate-Phosphate Buffer (pH 5.0):

Citric acid.1H₂O 7.3 g

Na₂HPO₄.2H₂O 11.86 g

Ad aq. dest. to 1|

Blocking Buffer:

Washing buffer +2% milk powder Plate outline

| | | 1 | 2 | 3 | 4 | 5 | 6 | 7 | 8 | 9 | 10 | 11 | 12 |
|---|---|---|---|---|---|---|---|---|---|---|---|---|---|
| Standards | A | 0 | 0 | 10 ng | 10 ng | 25 ng | 25 ng | 50 ng | 50 ng | 75 ng | 75 ng | 100 ng | 100 ng |
| Peptides | B | Pep1 | Pep1 | Pep1 | Pep1 | Pep1 | Pep1 | Pep1 | Pep1 | Pep1 | Pep1 | Pep1 | Pep1 |
| | C | Pep2 | Pep2 | Pep2 | Pep2 | Pep2 | Pep2 | Pep2 | Pep2 | Pep2 | Pep2 | Pep2 | Pep2 |
| | D | Pep3 | Pep3 | Pep3 | Pep3 | Pep3 | Pep3 | Pep3 | Pep3 | Pep3 | Pep3 | Pep3 | Pep3 |
| | E | Pep4 | Pep4 | Pep4 | Pep4 | Pep4 | Pep4 | Pep4 | Pep4 | Pep4 | Pep4 | Pep4 | Pep4 |
| | F | Pep5 | Pep5 | Pep5 | Pep5 | Pep5 | Pep5 | Pep5 | Pep5 | Pep5 | Pep5 | Pep5 | Pep5 |
| | G | Pep6 | Pep6 | Pep6 | Pep6 | Pep6 | Pep6 | Pep6 | Pep6 | Pep6 | Pep6 | Pep6 | Pep6 |
| Blanks | H | \acute{∅}Pep | \acute{∅} | \acute{∅} | \acute{∅} | \acute{∅} | \acute{∅} | \acute{∅} | \acute{∅} | \acute{∅} | \acute{∅} | \acute{∅} | \acute{∅} |
| | | Serum | Serum | Serum | Serum | Serum | Serum | Serum | Serum | Serum | Serum | Serum | Serum |
| | | 1 | 2 | 3 | 4 | 5 | 6 | 7 | 8 | 9 | 10 | 11 | 12 |

Diagnostic Potency of the Peptides

In the following tables, the diagnostic potency of the peptides for pregnancy complications and fertility diagnostics is illustrated exemplary in tables. The groups of patients, which were analysed comprise:

Apparently healthy female blood donors.

Patients presenting the first time for ivF in an ivF-facility. These patients did not show any endocrine or morphological reason for infertility. There was no male infertility involved in the fertility problem of the couple. No other autoantibody diseases such as aPL-syndrome or Lupus erythematodes were known or detected.

Patients after repetitive ivF-failure (two cycles without pregnancy). Not autoimmune disease known and nor endocrine, morphological or male reasons known for infertility.

Patients with an anamnesis of habitual abortion, wherein these patients had at least two consecutive abortions in the 1^{st} trimester of pregnancy.

Patients with an anamnesis of preeclampsia in at least one preceding pregnancy.

For optimal diagnostic sensitivity and specificity, the peptides can be combined to a small panel of diagnostic peptides. Abbreviations are as follows: MW (Mean); CI (confidence interval) C-off (cutoff value, defined as 1.1* (MW+CI)).

The Peptide shown in Table 1 exemplifies a peptide with a strong diagnostic profile in pregnancy complications, while infertility is detected much less sensitive.

TABLE 1

| | Female | | | | | |
|---|---|---|---|---|---|---|
| | donors, | | | | | |
| Peptide: | Blood | | Repetitive | Habitual | | |
| DQYIQQAHRSHI | Bank | IvF | ivF-failure | Abortion | Preeclampsia | |
| MW | 6.87 | 8.72 | 10.11 | 17.64 | 18.44 | |
| SD | 7.24 | 13.31 | 8.70 | 14.65 | 9.29 | |
| # of cases N | 33 | 15 | 25 | 17 | 14 | |
| 99% CI | 3.25 | 8.85 | 4.48 | 9.15 | 6.40 | |
| MW + CI | 10.12 | 17.57 | 14.59 | 26.79 | 24.84 | |
| MW − CI | 3.62 | −0.13 | 5.63 | 8.49 | 12.04 | |
| N > C-off | 10 | 3 | 13 | 11 | 12 | Number of cases above the Cut-OFF-level |
| Sensitivity | 0.30 | 0.20 | 0.52 | 0.65 | 0.86 | Fraction of those with the disease correctly |
| | | | | | | identified by the test |
| Specificity | 0.70 | 0.80 | 0.48 | 0.35 | 0.14 | Fraction of those without the disease correctly |
| | | | | | | identified by the test |

The peptide shown in table 2 has profile, which shows no strong preference of one of the patient groups, but gives a relatively constant, relatively low sensitivity profile through all groups.

TABLE 2

| | Female | | | | | |
|---|---|---|---|---|---|---|
| | donors, | | | | | |
| Peptide: | Blood | | Repetitive | Habitual | | |
| KQASNLTDMHYP | Bank | IvF | ivF-failure | Abortion | Preeclampsia | |
| MW | 2.57 | 4.95 | 7.87 | 11.52 | 8.53 | |
| SD | 3.45 | 3.84 | 10.69 | 12.25 | 10.80 | |
| # of cases N | 33 | 15 | 25 | 17 | 14 | |
| 99% CI | 1.55 | 2.55 | 5.51 | 7.65 | 7.44 | |
| MW + CI | 4.11 | 7.50 | 13.38 | 19.17 | 15.97 | |
| MW − CI | 1.02 | 2.39 | 2.36 | 3.87 | 1.09 | |
| N > C-off | 10 | 8 | 12 | 10 | 6 | Number of cases above the Cut-OFF-level |
| Sensitivity | 0.30 | 0.53 | 0.48 | 0.59 | 0.43 | Fraction of those with the disease correctly |
| | | | | | | identified by the test |
| Specificity | 0.70 | 0.47 | 0.52 | 0.41 | 0.57 | Fraction of those without the disease correctly |
| | | | | | | identified by the test |

The peptide in table 3 shows a strong profile in the field of infertile patients, while no diagnostic potency is present in the field of pregnancy complications (habitual abortion and preeclampsia).

TABLE 3

| | Female | | | | | |
|---|---|---|---|---|---|---|
| | donors, | | | | | |
| Peptide: | Blood | | Repetitive | Habitual | | |
| VYKSPNAYTLFS | Bank | IvF | ivF-failure | Abortion | Preeclampsia | |
| MW | 17.23 | 27.55 | 31.55 | 7.82 | 7.19 | |
| SD | 11.08 | 6.98 | 14.47 | 7.98 | 8.33 | |
| # of cases N | 33 | 14 | 25 | 51 | 42 | |
| 99% CI | 4.97 | 4.80 | 7.45 | 2.88 | 3.31 | |
| MW + CI | 22.20 | 32.35 | 39.00 | 10.70 | 10.50 | |
| MW − CI | 12.27 | 22.74 | 24.10 | 4.94 | 3.88 | |
| N > C-off | 11 | 12 | 21 | 4 | 4 | Number of cases above the Cut-OFF-level |
| Sensitivity | 0.33 | 0.86 | 0.84 | 0.08 | 0.10 | Fraction of those with the disease correctly |
| | | | | | | identified by the test |
| Specificity | 0.67 | 0.14 | 0.16 | 0.92 | 0.90 | Fraction of those without the disease correctly |
| | | | | | | identified by the test |

### FIGURES

FIG. 1 shows an example of an assay with sera from female blood bank donors, which form a reference group for the general female population. It is shown that two of the peptides demonstrate increased reactivity in patients with ivF (in vitro fertilization) failure and patients with habitual abortion.

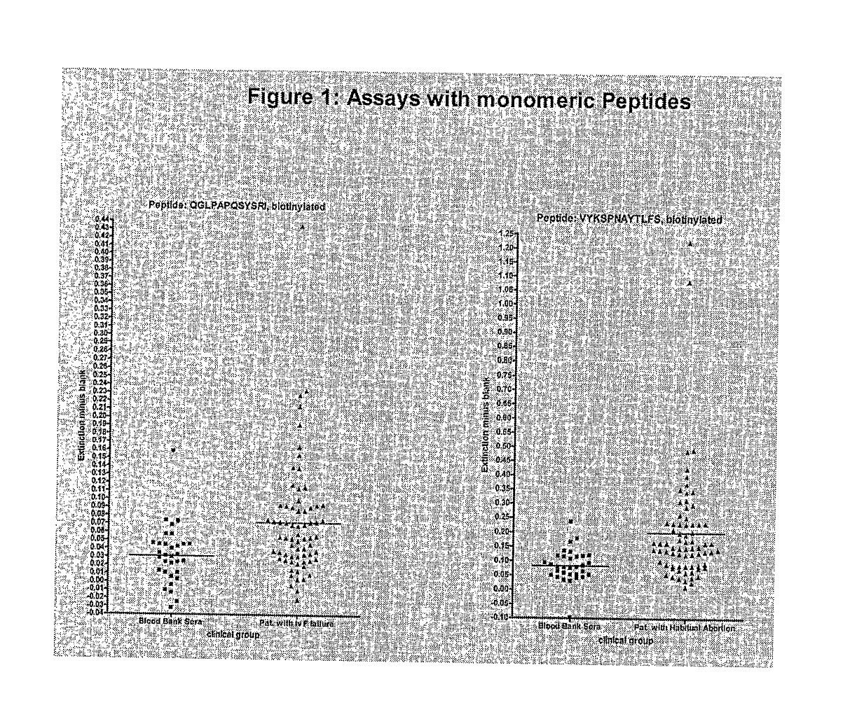

FIG. 2 shows that dimerisation of peptides using the protocol described in this text and using these dimeric peptides clearly increases the titers, which can be obtained in an ELISA-protocol.

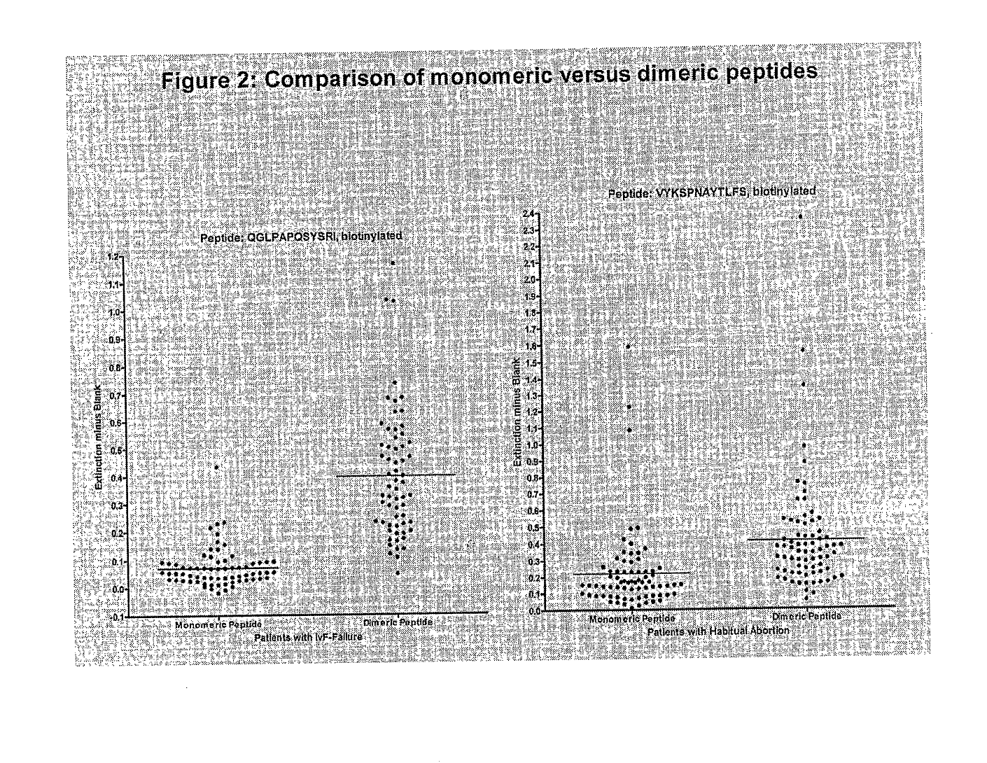

### REFERENCES

Beer A E, Kwak-Kim J Y, Beaman K D, Gilman-Sachs A (1998) Clinical utility of antiphospholipid antibodies? A negative study with power! Fertility and Sterility 69:166-168
Bermas B L, Schur P H, Kaplan M, Rose BD (1996a) Prognosis and therapy of the antiphospholipid antibody syndrome. Uptodate in Medicine 800:998-6374
Bermas B L, Schur P H, Rose BD (1996b) Clinical characteristics of the antiphospholipid antibody syndrome. Uptodate in Medicine 800
Bermas B L, Schur P H, Rose BD (1996c) Pathogenesis of the antiphospholipid antibody syndrome. Uptodate in Medicine 800
Bick R L, Baker W F (1999) Antiphospholipid syndrome and thrombosis. Seminars in Thrombosis and Hemostasis 25:333-350
Birdsall M A, Lockwood G M, Ledger W L, Johnson P M, Chamley L W (1996) Antiphospholipid antibodies in women having in-vitro fertilization. Human Reproduction 11:1185-1189
Check J H (1998) A negative study with power? Fertility and Sterility 70:599
Chilcott I T, Margara R, Cohen H, Rai R, Skull J, Pickering W, Regan L (2000) Pregnancy outcome is not affected by antiphospholipid antibody status in women referred for in vitro fertilization. Fertility and Sterility 73:526-530
Colaco C B, Male D K (1985) Anti-phospholipid antibodies in syphilis and a thrombotic subset of SLE: distinct profiles of epitope specificity. Clinical and Experimental Immunology 59:449-456
McIntyre J A, Wagenknecht D R, Faulk W P (2003) Antiphospholipid antibodies: discovery, definitions, detection and disease. Progress in Lipid Research 42:176-237

1. A method of assessing ex vivo the titre of fertility-relevant autoantibodies of a mammalian female, comprising the steps:
presentation of a molecule which binds to an idiotype of the fertility-relevant autoantibody, and
determining the amount of autoantibody being bound by said molecule from a sample of a body fluid of said mammalian female.

2. The method as claimed in claim 1, wherein said mammalian female is a human female.

3. The method as claimed in claim 1, wherein said body fluid is blood, serum, blood plasma or saliva.

4. The method as claimed in claim 1, wherein said molecule capable of binding to the idiotype of the fertility-relevant autoantibody is an oligo- or polypeptide.

5. The method as claimed in claim 4, wherein the oligopeptide is a peptide consisting of less than 40 amino acids.

6. The method as claimed in claim 4, wherein the polypeptide is a naturally occurring protein.

7. The method as claimed in claim 5, wherein the oligopeptide is part of the sequence of a naturally occurring polypeptide.

8. The method as claimed in claim 4, wherein the amino acid sequence of the polypeptides is selected from the sequences of pregnancy-associated plasma protein A (PAPP-A; gi:38045915) or from ADAM-TS 13 (gi:21265049).

9. The method as claimed in claim 1, wherein fertility-relevant autoantibodies being bound to the anti-idiotypic molecule are detected by an ELISA protocol using secondary antibodies being labelled with a detectable moiety such as an enzyme, a fluorochrome or biotin.

10. An oligopeptide selected from one of the sequences (SEQ ID No. 1-8)
VYKSPNAYTLFS (Seq ID No. 1)
QGLPAPQSYSRI (Seq ID No.5)
RPEPQGAYLEQG (Seq ID No. 2)
NSSYSPSLLESG (Seq ID No. 3)
DQYIQQAHRSHI (Seq ID No. 4)
KQASNLTDMHYP (Seq ID No. 6)
AQPNWTSLRSLP (Seq ID No. 7)
HVNPHLHVHAWD (Seq ID No. 8)
or retro-inverso derivatives of these peptide sequences.

11. Oligopeptides which are derivatives, fragments and/or homologues of the oligopeptides of claim 10, which differ from the oligopeptides in claim 10 such that
the oligopeptide amino acid sequence is extended at the amino-terminus and/or the carboxy-terminus by either up to 5 amino acids per terminus, and/or
in the homologous oligopeptides up to 3 amino acids are substituted by other amino acids, and/or
the fragments lack 1 or 2 amino acids at the N- or C-terminus, and/or
the oligopeptides are derivatized for detection by modifications including biotinylation, labelling by fluorochromes or radiolabelling;
or Retro-Inverso derivatives of these derivatives, fragments or homologues.

12. Oligopeptides comprising 8 to 50 amino acids which are fragments of the sequences of pregnancy-associated plasma protein A (PAPP-A; gi:38045915) or from ADAM-TS 13 (gi:21265049).

13. Dimers, trimers or multimers of oligopeptides of claim 10.

14. A medicament or pharmaceutical preparation comprising at least one oligopeptide of claim 10, derivatives, fragments and/or homologues thereof, dimers, trimers or multimers thereof, or a polypeptide having the sequence of pregnancy-associated plasma protein A (PAPP-A; gi:38045915) or from ADAM-TS13(gi:21265049), or a polypeptide comprising 8 to 50 amino acids which are fragments of the sequences of pregnancy-associated plasma protein A (PAPP-A; gi:38045915) or from ADAM-TS 13 (gi:21265049).

15. A diagnostic comprising at least one oligopeptide of claim 10, derivatives, fragments and/or homologues thereof, dimers, trimers or multimers thereof, or a polypeptide having the sequence of pregnancy-associated plasma protein A (PAPP-A; gi:38045915) or from ADAM-TS 13 (gi:21265049), or a polypeptide comprising 8 to 50 amino acids which are fragments of the sequences of pregnancy-associated plasma protein A (PAPP-A; gi:38045915) or from ADAM-TS 13 (gi:21265049).

16. A method for the treatment of fertility disorders, comprising administering the medicament of claim 14 to a patient in need thereof.

17. A method of diagnosing fertility disorders or pregnancy complications, comprising testing a patient with the diagnostic of claim 15.

18. The method of claim 4, wherein the oligo- or polypeptide is one of the sequences (SEQ ID No. 1-8)
VYKSPNAYTLFS (Seq ID No. 1)
OGLPAPQSYSRI (Seq ID No.5)
RPEPQGAYLEQG (Seq ID No. 2)
NSSYSPSLLESG (Seq ID No. 3)
DQYIQQAHRSHI (Seq ID No. 4)
KQASNLTDMHYP (Seq ID No. 6)
AQPNWTSLRSLP (Seq ID No. 7)
HVNPHLHVHAWD (Seq ID No. 8)
or retro-inverso derivatives of these peptide sequences, or derivatives, fragments and/or homologues thereof, or a dimer, trimer or multimer thereof, or a polypeptide having the sequence of pregnancy-associated plasma protein A (PAPP-A; gi:38045915) or from ADAM-TS13(gi:21265049), or a polypeptide comprising 8 to 50 amino acids which are fragments of the sequences of pregnancy-associated plasma protein A (PAPP-A; gi:38045915) or from ADAM-TS 13 (gi:21265049).

19. A diagnostic kit providing all reagents, standards, controls and accessories necessary to perform a method according to claim 1.

20. A method for contraception or inducing sterility comprising administering to a patient in need thereof a pharmaceutical preparation containing at least one oligopeptide of claim 10, derivatives, fragments and/or homologues thereof, dimers, trimers or multimers thereof, or a polypeptide having the sequence of pregnancy-associated plasma protein A (PAPP-A; gi:38045915) or from ADAM-TS 13 (gi:21265049), or a polypeptide comprising 8 to 50 amino acids which are fragments of the sequences of pregnancy-associated plasma protein A (PAPP-A; gi:38045915) or from ADAM-TS 13 (gi:21265049).

21. The method as claimed in claim 5, wherein the oligopeptide is a peptide consisting of less than 20 amino acids.

22. The method as claimed in claim 6, wherein the polypeptide is isolated from its natural source or expressed in a recombinant host system, the latter thereby including modifications of the sequence by addition of sequence TAG's to facilitate purification or detection of the protein.

23. Dimers, trimers or multimers of oligopeptides of claim 11.

24. Dimers, trimers or multimers of oligopeptides of claim 12.

25. A diagnostic kit providing all reagents, standards, controls and accessories necessary to perform a method according to claim 17.


SEQUENCE LISTING (8 sequences)

SEQ ID NO: 1 - protein, 12 aa, Artificial Sequence
  Description of Artificial Sequencesynthetic peptide
VYKSPNAYTLFS

SEQ ID NO: 2 - protein, 12 aa, Artificial Sequence
  Description of Artificial Sequencesynthetic peptide
RPEPQGAYLEQG

SEQ ID NO: 3 - protein, 12 aa, Artificial Sequence
  Description of Artificial Sequencesynthetic peptide
NSSYSPSLLESG

SEQ ID NO: 4 - protein, 12 aa, Artificial Sequence
  Description of Artificial Sequencesynthetic peptide
DQYIQQAHRSHI

SEQ ID NO: 5 - protein, 12 aa, Artificial Sequence
  Description of Artificial Sequencesynthetic peptide
QGLPAPQSYSRI

SEQ ID NO: 6 - protein, 12 aa, Artificial Sequence
  Description of Artificial Sequencesynthetic peptide
KQASNLTDMHYP

SEQ ID NO: 7 - protein, 12 aa, Artificial Sequence
  Description of Artificial Sequencesynthetic peptide
AQPNWTSLRSLP

SEQ ID NO: 8 - protein, 12 aa, Artificial Sequence
  Description of Artificial Sequencesynthetic peptide
HVNPHLHVHAWD